(19) United States
(12) Patent Application Publication
(10) Pub. No.: US 2019/0059832 A1
(43) Pub. Date: Feb. 28, 2019

(54) INDICATORS FOR RADIOLOGICAL PLACEMENT OF IMPLANTS

(71) Applicant: QUALCOMM Incorporated, San Diego, CA (US)

(72) Inventors: William Henry Von Novak, III, San Diego, CA (US); Seong Heon Jeong, San Diego, CA (US); Michael Harris, Carlsbad, CA (US)

(21) Appl. No.: 15/687,941

(22) Filed: Aug. 28, 2017

(51) Int. Cl. A61B 6/12; A61B 6/00; A61B 8/08; A61N 1/05; A61N 1/36; A61N 1/372; A61B 90/94; A61N 1/08

(52) U.S. Cl. CPC A61B 6/12; A61B 6/501; A61B 8/0841; A61B 8/0808; A61N 1/0534; A61B 2090/3925; A61B 6/487; A61N 1/36132; A61N 1/37217; A61B 90/94; A61N 1/08; A61N 1/36067

(57) ABSTRACT

Disclosed herein are techniques for providing a status of an implant device during or after the implantation of the implant device using reconfigurable indicators. In one embodiment, an implant device includes a reconfigurable indicator and a controller comprising or coupled to the reconfigurable indicator. The reconfigurable indicator is implantable, and is reconfigurable to a plurality of states by the controller. The reconfigurable indicator is configured to be visible by an imaging system when the reconfigurable indicator is implanted and set to a first state of the plurality of states.

### BACKGROUND

Medical implant devices are widely used today. These medical implant devices are often electrical in nature. For example, many medical implant devices use electrodes implanted in areas of interest inside a human body to sense electrical activities and/or apply electrical stimulus signals for treatment. In most cases, it is important that the electrodes for the medical implants are placed correctly in the areas of interest and remain in the areas of interest for the lifetime of the medical implants.

### BRIEF SUMMARY

Techniques disclosed herein relate to a dynamically reconfigurable and radiologically visible indicator within a medical implant device for providing information regarding the operation status of the location of the medical implant device, such as a location of an electrode of the medical implant device, when viewed using a noninvasive imaging system, such as a fluoroscope or an ultrasonic imager, during or after an implantation surgery.

In accordance with an example implementation, an implant device may include a reconfigurable indicator and a controller including or coupled to the reconfigurable indicator. The reconfigurable indicator may be implantable, and may be reconfigurable to a plurality of states by the controller. The reconfigurable indicator may be configured to be visible by an imaging system when the reconfigurable indicator is implanted and set to a first state of the plurality of states. In some implementations, the reconfigurable indicator may be visible by a radiological imaging system or an ultrasonic imaging system when set to the first state.

In some implementations of the implant device, the imaging system may include an X-ray imaging system, and the reconfigurable indicator may include a symbol including a radiopaque material, where the symbol may be configured to at least partially block X-rays from the X-ray imaging system in the first state of the plurality of states. In some implementations, the symbol on the reconfigurable indicator may include at least one of a letter, a number, or a figure. In some implementations, the controller may include two or more reconfigurable indicators, where the two or more reconfigurable indicators may be configurable to, in combination, indicate a status of the implant device. In some implementations, in a second state of the plurality of states, the symbol on the reconfigurable indicator may be configured to be invisible by the X-ray imaging system.

In some implementations of the implant device, each state of the plurality of states of the reconfigurable indicator may correspond to a different status of the implant device. The reconfigurable indicator, when set to a different state of the plurality of states, may be configured to cause a different image of the reconfigurable indicator to be formed by the imaging system. In some implementations, the controller may include instructions stored thereon. The instructions, when executed by the controller, may cause the controller to receive electrical signals from an implantable electrode of the implant device, determine a location of the implantable electrode based on the electrical signals, and set the reconfigurable indicator to a state of the plurality of states based on the determined location of the implantable electrode. In some implementations, the instructions may further cause the controller to determine a status of the implant device based on the electrical signals, and set the reconfigurable indicator to indicate the determined status of the implant device. In some implementations, the implant device may include a deep brain stimulation (DBS) device and an implantable electrode, where the implantable electrode may be configured to collect electrical signals in a brain of a person. The instructions, when executed by the controller, may further cause the controller to detect a characteristic pattern of electrical pulses associated with a disease (e.g., Parkinson's disease) from the electrical signals collected by the implantable electrode, and set the reconfigurable indicator to a state of the plurality of states to indicate that the implantable electrode is in an area of interest in the brain of the person.

In some implementations of the implant device, the controller may further include an actuator configured to shift or rotate the reconfigurable indicator to set the reconfigurable indicator to the plurality of states. The actuator may include at least one of a magnetic, electrostatic, or electromechanical actuator. In some implementations, the imaging system may include a fluoroscope or an ultrasonic imager.

In some implementations, the controller may be configured to be implantable under a scalp of a person. In some implementations, the controller may include an enclosure and the reconfigurable indicator may be positioned within the enclosure. In some implementations, the enclosure may be vacuumed or may be filled with a fluid.

In some implementations, the imaging system may include an ultrasonic imaging system, and the reconfigurable indicator may include a solenoid. In the first state of the plurality of states, the solenoid may be configured to extend and support a surface of the controller. In some implementations, the imaging system may include an ultrasonic imaging system, and the reconfigurable indicator may include an ultrasonic transponder. In the first state of the plurality of states, the ultrasonic transponder may be configured to detect an incoming ultrasonic signal from the ultrasonic imaging system, and, in response to detecting the incoming ultrasonic signal, transmit a return ultrasonic signal. In some embodiments, the return ultrasonic signal may have a predetermined amplitude. In some embodiments, the return ultrasonic signal may be transmitted at a predetermined time after the incoming ultrasonic signal is received by the ultrasonic transponder. In some embodiments, the return ultrasonic signal may include two or more ultrasonic pulses, and a delay between two of the two or more ultrasonic pulses is a predetermined value.

In accordance with an example implementation, a method for indicating a status of an implant device is provided. The method may include receiving electrical signals collected by an implantable electrode, determining the status of the implant device based on the electrical signals, and setting a reconfigurable indicator to a state from a plurality of states to indicate the determined status of the implant device. The reconfigurable indicator may be implantable and may be reconfigurable to the plurality of states. The reconfigurable indicator may be configured to be visible by an imaging system when the reconfigurable indicator is implanted and set to a first state of the plurality of states.

In some implementations of the method, each state of the plurality of states of the reconfigurable indicator may correspond to a different status of the implant device. The reconfigurable indicator, when set to a different state of the plurality of states, may be configured to cause a different image of the reconfigurable indicator to be formed by the imaging system. In some implementations, determining the status of the implant device may include determining a location of the implantable electrode. In some implementations, setting the reconfigurable indicator to the state may include shifting or rotating the reconfigurable indicator using an actuator, such as a magnetic, electrostatic, or electromechanical actuator.

In some implementations, the method may further include reading, using the imaging system, the determined status of the implant device indicated by the reconfigurable indicator. The imaging system may include an X-ray imaging system, and the reconfigurable indicator may include a symbol including a radiopaque material, where, in the first state of the plurality of states, the symbol may be configured to at least partially block X-rays from the X-ray imaging system. In some embodiments, determining the status of the implant device based on the electrical signals may include detecting a characteristic pattern of electrical pulses associated with a disease (e.g., Parkinson's disease) from the electrical signals.

In some implementations, the imaging system may include an ultrasonic imaging system, the reconfigurable indicator may include a solenoid, and setting the reconfigurable indicator to the first state may include causing the solenoid to extend. In some implementations, the imaging system may include an ultrasonic imaging system, the reconfigurable indicator may include an ultrasonic transponder. Setting the reconfigurable indicator to the first state may include causing the ultrasonic transponder to detect an incoming ultrasonic signal from the ultrasonic imaging system, and, in response to detecting the incoming ultrasonic signal, transmit a return ultrasonic signal including one or more ultrasonic pulses.

In accordance with another example implementation, an apparatus may be provided, which may include means for receiving electrical signals collected by an implantable electrode, means for determining a status of the apparatus based on the electrical signals, and means for indicating the determined status of the apparatus. The means for indicating the determined status of the apparatus may be implantable and may be reconfigurable to a plurality of states, where each state of the plurality of states may correspond to a different status of the apparatus. The means for indicating the determined status of the apparatus may be configured to be visible by an imaging system when the means for indicating the determined status of the apparatus is implanted and set to a first state of the plurality of states.

In some implementations of the apparatus, the means for indicating the determined status of the apparatus, when set to a different state of the plurality of states, may be configured to cause a different image of the means for indicating the determined status of the apparatus to be formed by the imaging system. In some implementations, the means for determining the status of the apparatus based on the electrical signals may include means for determining a location of the implantable electrode based on the electrical signals. In some implementations, the means for indicating the determined status of the apparatus may be movable and may include a symbol including a radiopaque material. The imaging system may include an X-ray imaging system, and the symbol may be configured to at least partially block X-rays from the X-ray imaging system in the first state of the plurality of states.

In accordance with yet another example implementation, a non-transitory computer-readable storage medium including machine-readable instructions stored thereon is disclosed. The non-transitory computer-readable storage medium may include instructions that, when executed by one or more processors, may cause the one or more processors to receive electrical signals collected by an implantable electrode of an implant device, determine a status of the implant device based on the electrical signals, and set a reconfigurable indicator to a state from a plurality of states to indicate the determined status of the implant device. The reconfigurable indicator may be implantable and may be reconfigurable to the plurality of states. The reconfigurable indicator may be configured to be visible by an imaging system when the reconfigurable indicator is implanted and is set to a first state of the plurality of states. In some implementations, determining the status of the implant device based on the electrical signals may include detecting a characteristic pattern of electrical pulses associated with a disease from the electrical signals collected by the implantable electrode.

### BRIEF DESCRIPTION OF THE DRAWINGS

Aspects of the present disclosure are illustrated by way of example. Non-limiting and non-exhaustive aspects are described with reference to the following figures, wherein like reference numerals refer to like parts throughout the various figures unless otherwise specified.

FIG. 1 illustrates an example deep brain stimulation system implanted in a patient's body.

FIG. 2 illustrates an example deep brain stimulation implant system, according to certain aspects of the present disclosure.

FIG. 3 is a simplified block diagram of an example controller of a deep brain stimulation system, according to certain aspects of the present disclosure.

FIG. 4A illustrates example electrical signals generated by electrical activities in a healthy person's brain.

FIG. 4B illustrates example electrical signals generated by electrical activities in the brain of a patient suffering from Parkinson's disease.

FIG. 5A illustrates example radiological indicators set in “OFF” states, according to certain aspects of the present disclosure.

FIG. 5B illustrates example radiological indicators set in “ON” states, according to certain aspects of the present disclosure.

FIG. 6 is an illustrative X-ray image of a patient's head with an implanted deep brain stimulation implant system, according to some aspects of the present disclosure.

FIG. 7 illustrates an example implant device in the abdomen of a patient, according to certain aspects of the present disclosure.

FIG. 8 is a flow chart illustrating an example method for indicating a status of an implant device, according to some aspects of the present disclosure.

FIG. 9 is a block diagram of an example wireless device for implementing some of the examples described herein.

### DETAILED DESCRIPTION

Several illustrative embodiments will now be described with respect to the accompanying drawings, which form a part hereof. The ensuing description provides embodiment(s) only and is not intended to limit the scope, applicability, or configuration of the disclosure. Rather, the ensuing description of the embodiment(s) will provide those skilled in the art with an enabling description for implementing an embodiment. It is understood that various changes may be made in the function and arrangement of elements without departing from the spirit and scope of this disclosure.

Medical implants are widely used today. Many medical implant devices help extend and improve quality of life. These medical implants are often electrical in nature. For example, many medical implants, such as deep brain stimulation (DBS) devices, implantable cardioverter defibrillators (ICDs), and pacemakers, use electrodes implanted in areas of interest inside a human body to sense electrical activities occurring in the areas of interest and/or apply electrical stimulus signals to the areas of interest for treatment. In most cases, it is important that the electrodes for the medical implants are placed correctly in the areas of interest and remain in the areas of interest for the lifetime of the medical implants.

Techniques disclosed herein relate to using a reconfigurable, radiologically visible indicator within a medical implant device to provide information regarding the location of an electrode of the medical implant device and/or the operational status of the medical implant device using a noninvasive imaging system, such as a fluoroscope or an ultrasonic imager, during or after an implantation surgery. No additional external monitoring device and hence no physical connection to and/or disconnection from an external monitoring device during the implantation surgery may be needed. As such, the risk that the electrode may be unintentionally moved during the implantation surgery may be significantly reduced. Furthermore, the location of the electrode and/or the operational status of the medical implant device may be constantly monitored during the lifetime of the medical implant using the noninvasive imaging system.

Although many of the following embodiments are described with respect to a DBS system, those skilled in the art would understand that the techniques disclosed herein can be used in many types of medical implants, including, but not limited to, for example, DBS devices, ICDs, and pacemakers.

### I. Deep Brain Stimulation

Deep brain stimulation (DBS) is a surgery to implant a device that sends electrical signals through implantable electrodes to areas of the brain responsible for body movement. The electrodes are placed deep in the brain and are connected to a stimulator device—a neurostimulator. Similar to a heart pacemaker, a neurostimulator uses electrical pulses to regulate brain activities. A DBS system regulates abnormal electrical signaling patterns in the brain. In order to control movement and other functions, brain cells communicate with each other using electrical signals. In Parkinson's disease, these electrical signals become irregular and uncoordinated and lead to motor symptoms. A DBS system interrupts the atypical signaling patterns in a way that allows the cells to communicate more smoothly, thereby lessening the symptoms. A DBS system can help to reduce symptoms, such as tremor, slowness of movement, stiffness, and walking problems caused by Parkinson's disease, dystonia, or essential tremor. A successful DBS implantation allows people to better manage their symptoms, reduce their medications, and improve their quality of life.

A. DBS System

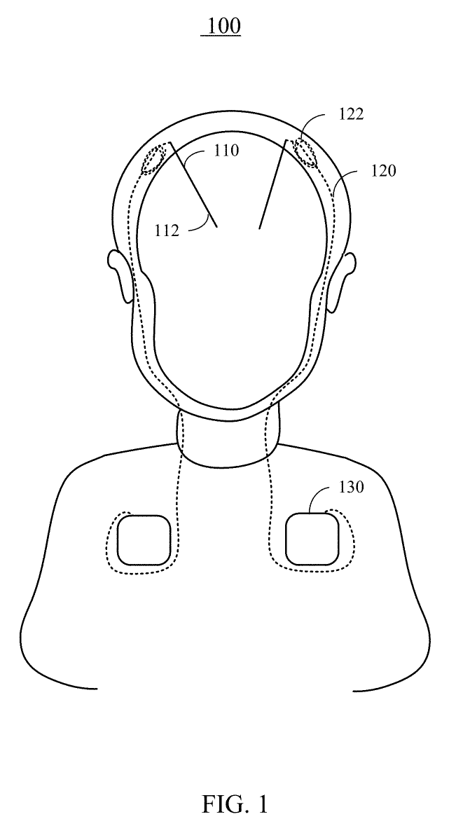

FIG. 1 illustrates an example deep brain stimulation system 100 implanted in a patient's body. DBS system 100 includes three components that are all implanted inside a human body: a lead 110, an extension wire 120, and a controller 130. Controller 130 is a programmable battery-powered pacemaker device that generates electrical pulses to interfere with the neural activities in target areas of the brain. Controller 130 is generally placed under the skin of the chest below the collarbone or lower in the chest, or under the skin over the abdomen. In a DBS system, the controller may also be referred to as a neurostimulator, an implanted pulse generator (IPG), or simply a stimulator. When turned on, controller 130 sends electrical pulses to block the faulty nerve signals that may cause tremors, rigidity, and other symptoms. Lead 110, sometimes referred to as an electrode, is an insulated wire (e.g., coated with polyurethane) with one or more electrodes 112 (e.g., platinum-iridium electrodes) at the tip that are placed in one or more different nuclei of the brain to deliver electric pulses to the brain tissue. Electrode 112 may be placed inside the brain and may be connected to extension wire 120 through a small hole in the skull. Extension wire 120 is an insulated wire that connects lead 110 to controller 130. Extension wire 120 is typically placed under the skin and runs from the scalp, behind the ear, down the side of the neck, and to the chest. In some cases, a coil of wire 122 is left under the scalp for attachment to extension wire 120. A patient may use a handheld controller to turn the DBS system on and off. Medical personnel may program the stimulation settings of the stimulator with a wireless device. The stimulation settings can be adjusted as a patient's condition changes over time.

In deep brain stimulation, the electrodes are placed in specific areas of the brain (usually the subthalamic nucleus) depending on the symptoms being treated. For example, for dystonia, dyskinesia, and other symptoms associated with Parkinson's disease (e.g., rigidity, slowness, bradykinesia/akinesia, and tremor caused by the death of dopamine-producing nerve cells responsible for relaying the message that controls body movement), the electrode may be placed in either the globus pallidus internus (GPI) or the subthalamic nucleus (STN). For non-Parkinsonian essential tremor (e.g., involuntary rhythmic tremors of the hands and arms occurring both at rest and during purposeful movement), the electrode may be placed in the ventrointermediate nucleus (VIM) of the thalamus. For obsessive-compulsive disorder or depression (OCD), the electrode may be placed in the nucleus accumbens. For incessant pain, the electrode may be placed in the posterior thalamic region or periaqueductal gray. For Parkinson plus patients, the electrode may be placed in two nuclei, subthalamic nucleus and tegmental nucleus of pons, with the use of two pulse generators simultaneously. For epilepsy treatment, the electrode may be placed in the anterior thalamic nucleus. Because the left side of the brain controls the right side of the body and vice versa, DBS is commonly performed on both sides of the brain. The electrodes may be placed on both the left and right sides of the brain through small holes made at the top of the skull.

B. Surgical Procedure

As described above, all three components of a DBS system are surgically implanted inside the body. Lead implantation may take place under local anesthesia (“awake DBS procedure”) or with the patient under general anesthesia (“asleep DBS procedure”). A hole (e.g., about 14 mm in diameter) may be drilled in the skull and a probe electrode (also referred to as recording electrode) may be inserted stereotactically. During the awake DBS procedure with local anesthesia, feedback from the patient may be used to determine the optimal placement for a permanent electrode. During the asleep DBS procedure, intraoperative magnetic resonance imaging (MRI) guidance may be used for direct visualization of brain tissue and the electrode. The installation of the controller and the extension wire may occur under general anesthesia after the lead implantation.

The basic DBS surgical method is called frame-based stereotaxis, which is the traditional method for approaching deep brain targets though a small skull opening. The frame-based stereotaxis may be performed with the patient awake, secured in a rigid head-frame, and off from medication. Local anesthetic is used to numb the skin where the incision is made. Brain mapping through a recording electrode may be used to determine whether an electrode is in the optimal location, and a stimulating electrode may then replace the recording electrode and be turned on to test for efficacy and to evaluate for possible side effects.

During the surgery, the rigid frame is attached to the patient's head. A brain imaging study may be obtained with the frame in place. The images of the brain and frame may be used to calculate the position of the desired brain target and guide instruments to the target with minimal trauma to the brain. A surgeon may use MRI and/or computerized tomography (CT) images and special computer software to plan the trajectory of the electrode. An incision may then be made on top of the head behind the hairline and a small opening may be made in the skull. If both sides of the brain are to be implanted, the skull opening is made on both sides.

Brain mapping using thin recording electrodes is then performed to record brain cell activities to confirm that the recording electrodes are in the intended target areas, or to make fine adjustments (e.g., within about 2 millimeters) in the intended target areas to find the optimal location. Usually a patient remains awake during surgery so that the patient can answer questions and perform certain tasks (e.g., lift arms or legs or count numbers) to make sure that the recording electrodes are positioned correctly. The brain's electrical signals may be played over a speaker or may be displayed using other external monitoring instruments (e.g., a computer monitor for waveform display) so that the surgical team can listen or look for distinctive patterns of neuronal activity that indicate the location of the recording electrode. Since each person's brain is different, the time it takes for the mapping may vary from about 30 minutes to up to about two hours for each side of the brain.

The prospect of being awake during brain surgery concerns some patients, as does the requirement to be off medication. Asleep DBS eliminates the need for awake brain mapping because real-time MRI scanning may be used to directly visualize brain tissue and the electrode, locate the target areas, determine the correct implantation trajectory, and confirm final electrode placement. For example, the electrode of the DBS implant system may be installed using a stereotactic technique, such as using a fluoroscope, to guide the placement of the electrode, where a radiopaque tip of the electrode may be placed in an area of interest in the brain based on the visible cranial structures. Electrical activities of neurons in the brain, such as electrical activities associated with tremors in Parkinson's disease, can then be collected by the electrode and monitored to determine whether the electrode is placed correctly. For example, when the electrode is properly placed, it may collect a characteristic pattern of electrical pulses associated with tremor generation in Parkinson's disease generated by neurons in the area of interest.

When the correct target site is confirmed with the recording electrode, a permanent DBS electrode may be inserted to replace the recording electrode and tested. The testing does not focus on relief of motor symptoms but rather on unwanted stimulation-induced side effects. This is because the beneficial effects of stimulation may take hours or days to develop, whereas unwanted effects may be present immediately. For the testing, the device may be deliberately tuned to an intensity level higher than what is normally used, in order to deliberately produce unwanted stimulation-induced side effects (such as tingling in the arm or leg, difficulty speaking, or a pulling sensation in the tongue or face).

A few weeks after surgery, a movement disorder specialist may use a handheld programmer to set parameters tailored to each patient's unique symptoms into the controller. The DBS settings may be gradually tweaked over time and medications may be simultaneously adjusted. Determining the optimal combination of medications and DBS settings that gives the most benefit and the least side effects may take several months or even a year.

### II. Radiological Indicators

As described above, currently, the electrical activities collected by the electrode are generally monitored by connecting the electrode to a large external monitoring device separate from the controller, such as an oscilloscope or a speaker. This generally involves connecting the distal end of the electrode to the external monitoring device, verifying that the placement is appropriate based on the monitored signals, disconnecting the electrode from the external monitoring device, and re-routing the distal end of the electrode to the controller through the extension wire. In some cases where a recording electrode is used to find the area of interest, the recording electrode is replaced with the permanent DBS electrode after the area of interest is located. If the electrode moves, for example, during disconnecting the electrode from the external monitoring device and/or re-routing the distal end of the electrode to the controller, or the permanent DBS electrode is not at the location verified using the recording electrode, the final position of the electrode may be incorrect, but the surgeon may not know that the electrode has been moved. In addition, some components of an implant device, such as a medical implantable connector, may be a common source of failure because, for example, the connector may not be fully hermetically sealed. Therefore, it is important to constantly monitor the electrical activities collected by the electrode during and after the implantation procedure to ensure the correct location of the electrode and the proper connection of the implant system.

Some visible indicators, such as light-emitting diodes (LEDs), can be used on the controller to indicate the correct location of the electrode and the proper connection and operation of the implant system. However, in many cases, an LED may not be visible to the surgeon. Wireless links, such as a Bluetooth low energy (BLE) link, may be used to provide data to the surgeon. However, the power consumption and reliability of the wireless links may not meet the requirements of the implant system. Wired links, such as an external indicator light attached to the controller by a wire that can be removed before the end of surgery, may be used in some cases. However, using the wired links may have the same drawbacks as using the external monitoring device discussed above.

Techniques disclosed herein use a dynamically reconfigurable and radiologically visible indicator within a medical implant to provide information regarding the location of the electrode when viewed using a noninvasive imaging system, such as a fluoroscope or an ultrasonic imager, during or after an implantation surgery.

As described above, during a DBS surgery, a controller of the DBS implant system may be implanted subcutaneously under the scalp, and an electrode of the DBS implant system may be installed using stereotactic techniques, such as a fluoroscope, where a radiopaque electrode tip may be placed in relation to the visible cranial structures of the brain. Electrical activities of neurons collected by the electrode, such as electrical activities associated with tremors in Parkinson's disease, can be monitored to determine whether the electrode is placed correctly. For example, when the electrode is properly placed, it may detect a characteristic pattern of electrical pulses generated by brain neurons that indicates a tremor generation associated with Parkinson's disease. Since, during the implantation of a neuroceutical implant system, a fluoroscope (i.e., a real time X-ray imaging system) or ultrasound imager is often used to guide the placement of the electrode, a radiologically visible indicator (one visible on a fluoroscope or an ultrasound imager) that is controlled by a controller of the implant system coupled to the electrode can be used to provide the desired information in real-time when the fluoroscope or the ultrasound imager is used during the implantation. As such, no additional external monitoring device may be needed to monitor the condition of the electrode by monitoring the electrical signals collected by the electrode. Furthermore, because the electrode is not physically connected to an external monitoring device for signal monitoring, there is no need to physically disconnect the electrode from the external monitoring device in order to connect the electrode to the controller, and thus the risk that the electrode may be implanted in a wrong location or may be moved during or after the implantation procedure may be significantly reduced.

It is noted that although embodiments described herein use a fluoroscope (e.g., for DBS) or an ultrasound imager (e.g., for abdomen implantation), techniques disclosed herein may be used to monitor any medical implantation in any part of a body in real-time using any radiological, ultrasonic, or other noninvasive imaging technique.

According to one embodiment of the present invention, rather than using an external monitoring device to monitor the electrical activities collected by the electrode, one or more radiologic indicators connected to the controller of the implant system may be used to provide information regarding the electrical activities collected by the electrode and/or whether the electrode remains in the area of interest. Therefore, the electrode may be permanently connected to the controller during the implantation procedure, rather than being connected to an external monitoring device for identifying the area of interest, disconnected from the external monitoring device after the area of interest is located, and re-routed to the controller.

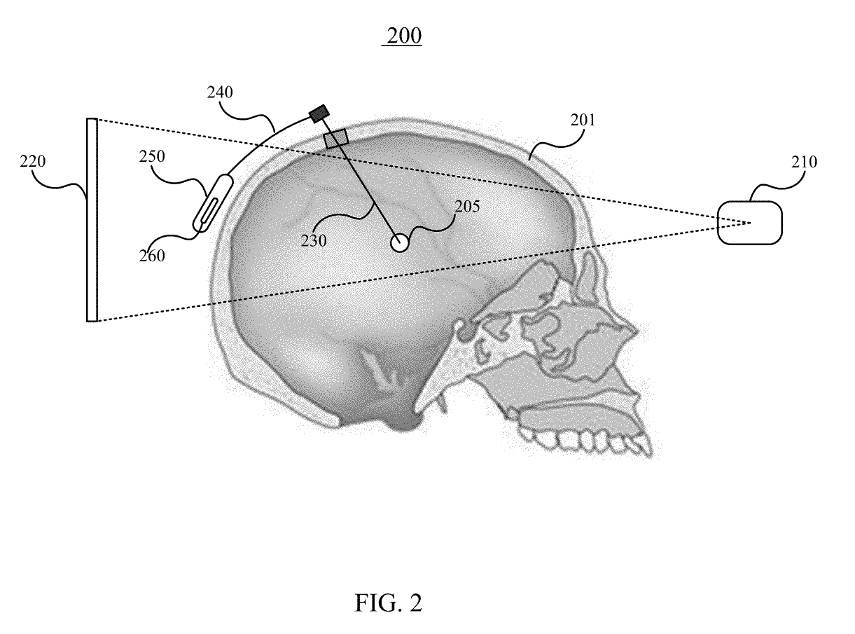

FIG. 2 illustrates an example DBS implant system 200, according to certain aspects of the present disclosure. DBS implant system 200 includes an electrode (or lead) 230, an extension wire 240, and a controller 250. During the implantation procedure, controller 250 may be implanted subcutaneously under the scalp (not shown in FIG. 2) and above the skull 201 of a patient. Controller 250 may include a neurostimulator to deliver electrical stimulation pulses to target areas of the brain that control the body movement to block abnormal nerve signals that may be responsible for causing tremor and other Parkinson's disease symptoms, such as rigidity, stiffness, slowed movement, and walking problems. Controller 250 may also include one or more indicators 260 for indicating the status of the DBS implant system. In some cases, indicator(s) 260 may be separate from controller 250 and may be electrically coupled to controller 250. In some cases, at least a portion of controller 250 may be implanted under the skin near the collarbone, in the chest, or under the skin over the abdomen of the patient.

Electrode 230 is connected to controller 250 through extension wire 240 that includes an insulated wire passing under the scalp. Electrode 230 is then inserted through a small opening in skull 201 and implanted in an area of interest 205 in the brain of the patient, such as the GPI or STN. Electrode 230 may collect electrical signals generated by electrical activities in area of interest 205 and send the electrical signals to controller 250. Electrode 230 may include a tip that is radiopaque, and thus can be seen using a radiological imaging system.

During the placement of electrode 230, a real-time X-ray imaging system (e.g., a fluoroscope) may be used to monitor the location of the radiopaque tip of electrode 230 with respect to the visible cranial structures of skull 201. The X-ray imaging system may include an X-ray source 210 and an X-ray sensor 220 arranged on opposite sides of the skull such that the tip of electrode 230 may be in the field of view of the X-ray imaging system and may be detected by the X-ray imaging system. Controller 250 may also be in the field of view of the X-ray imaging system such that indicator(s) 260 may be detected using the X-ray imaging system.

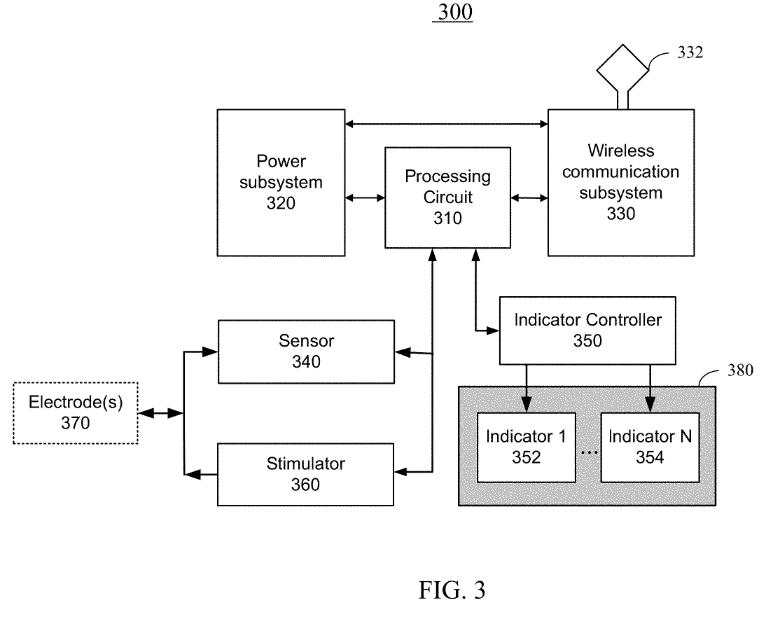

FIG. 3 is a simplified block diagram of an example controller 300 of a DBS system, according to certain aspects of the present application. Controller 300 may be used as controller 250 of FIG. 2. Controller 300 may include a processing circuit 310, a power subsystem 320, and a wireless communication subsystem 330.

Power subsystem 320 may include one or more rechargeable or non-rechargeable batteries, such as alkaline batteries, lead-acid batteries, lithium-ion batteries, zinc-carbon batteries, and NiCd or NiMH batteries. Power subsystem 320 may also include one or more power management circuits, such as voltage regulators, DC-to-DC converters, wireless charging circuits, or energy harvest circuits, etc. In some embodiments, power subsystem 320 may include a real-time clock using, for example, a watch crystal or other crystals.

Wireless communication subsystem 330 may include one or more of a cellular communication subsystem, a Wi-Fi transceiver, a Bluetooth, BLE, or ZigBee transceiver, or other wireless communication subsystems, such as a near-field communication (NFC) subsystem. Wireless communication subsystem 330 may be connected to or include one or more antennas 332, such as a printed antenna (e.g., a microstrip or patch antenna) or an antenna array. Wireless communication subsystem 330 may be operable to be powered on, powered off, or in a standby (i.e., sleep) mode. When powered on, wireless communication subsystem 330 may communicate with an external device for configuring the setting of controller 300, such as setting various parameters of controller 300. When powered off, circuits in wireless communication subsystem 330 may consume no power. When in a standby mode, only a small portion of wireless communication subsystem 330 may be activated, while the rest of wireless communication subsystem 330 may be deactivated or powered off. For example, in some embodiments, a wireless receiver, a wireless signal detector, or a wireless sniffer may remain powered on for receiving instructions from a patient or medical personal to turn on the DBS system, and other circuits may be powered off

Processing circuit 310 may include a processor (e.g., ARM® or MIPS® processor), a microcontroller, or an application specific integrated circuit (ASIC). Processing circuit 310 may control the operations of the DBS system and other components of controller 300. For example, processing circuit 310 may receive electrical signals generated by electrical activities in areas of interest in the patient's brain from a sensor 340, which may be connected to one or more electrodes 370 that are implanted in the areas of interest of the patient's brain to collect the electrical signals. Sensor 340 may include circuits such as amplifier(s), filter(s), analog-to-digital converter(s), etc., and may convert electrical signals generated by the electrical activities in the areas of interest in the patient's brain into digital signals that can be processed by processing circuit 310 for determining whether electrode(s) 370 are in appropriate locations. For example, processing circuit 310 may analyze the waveforms of the collected electrical signals and determine whether a characteristic pattern of electrical pulses generated by brain neurons associated with Parkinson's disease is present in the collected electrical signals.

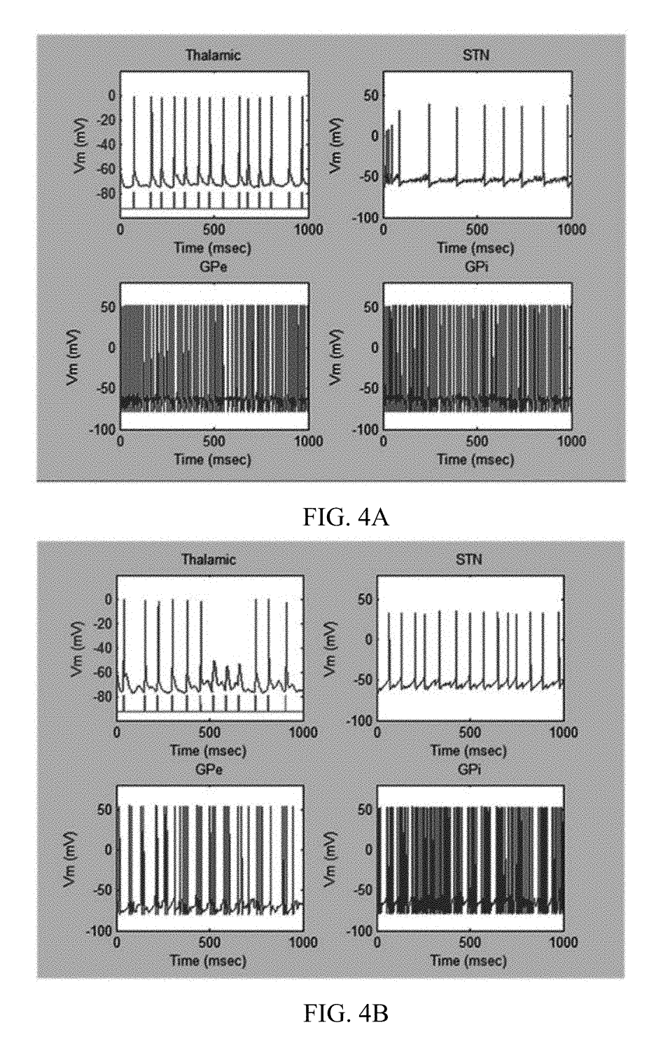

FIG. 4A illustrates example electrical signals generated by electrical activities in a healthy person's brain. FIG. 4A shows periodic electrical pulse sequences generated in four areas of the brain of a healthy person under normal conditions: the thalamic area, the subthalamic nucleus (STN), the external globus pallidus (GPe), and the medial globus pallidus internus (GPI), which may be helpful for understanding different stages of Parkinson's disease. See, e.g., K. Yue et al., “Magneto-Electric Nano-Particles for Non-Invasive Brain Stimulation,” PLOS ONE 7(9) (2012). As can be seen from FIG. 4A, in electrical signals collected from the brain of a healthy person, all electrical pulses are periodic and uniform in amplitude, and no lapses in the periodic sequence are present.

FIG. 4B illustrates example electrical signals generated by electrical activities in the brain of a patient suffering from Parkinson's disease. FIG. 4B shows typical electrical signals collected from the brain of the patient suffering from Parkinson's disease at the same four areas as in FIG. 4A. It can be seen from FIGS. 4A and 4B that the most drastic difference between the brain electrical signals of a healthy person and the brain electrical signals of a patient suffering from Parkinson's disease is an appearance of pronounced lapses in the periodic pulsed sequences, in particular, in the thalamic region of the patient suffering from Parkinson's disease. The periodicity of the pulses in other regions of the brain of the patient suffering from Parkinson's disease may be broken as well.

Thus, by comparing the detected electrical signals collected by electrode(s) 370 against electrical signals generated by electrical activities in the brain of a patient suffering from Parkinson's disease and/or electrical signals generated by electrical activities in the brain of a healthy person as shown in FIGS. 4A and 4B, processing circuit 310 may determine whether electrode(s) 370 are at areas of interest in the patient's brain. In some implementations, processing circuit 310 may analyze the detected electrical signals by extracting the frequency spectrum and/or amplitude information of signals at different frequency (e.g., using fast Fourier transform or other techniques) to determine whether a characteristic pattern of electrical pulses generated by brain neurons associated with Parkinson's disease is present in the detected electrical signals. Processing circuit 310 may determine that an electrode is at an area of interest when the characteristic pattern is present in the electrical signals collected by the electrode or the electrical signals collected by the electrode match with typical electrical activities in the brain of a patient suffering from Parkinson's disease.

During the placement of electrode(s) 370, processing circuit 310 may send instructions to an indicator controller 350, which may control one or more indicators, such as indicator 1 (352), . . . , and indicator N (354). In some embodiments, the indicators may be positioned inside an enclosure 380 that is vacuumed or is filled with a fluid, such as air or a liquid, and may be movable within enclosure 380. The indicators may be radiological indictors that can be set, actuated, or otherwise reconfigured by indicator controller 350 based on the instructions from processing circuit 310 to indicate the status of the electrode. When reconfigured by indicator controller 350, the indicators may become visible or invisible by a real-time X-ray imaging system as shown in FIG. 2. For example, in some embodiments, when processing circuit 310 determines that an electrode 370 is at an area of interest, indicator controller 350 may reconfigure the corresponding indicator(s) such that the corresponding indicator(s) become visible using the real-time X-ray imaging system or present a particular symbol or code on the display of the X-ray imaging system. Based on the image of the indicator(s) in the images of the X-ray imaging system, a surgeon may know whether the electrode is at the area of interest.

As shown in FIG. 3, controller 300 may also include a stimulator 360 that is connected to one or more electrodes 370. During the placement of electrode(s) 370, processing circuit 310 may control stimulator 360, for example, to test for efficacy and to evaluate for side effects. After the surgery, processing circuit 310 may control stimulator 360 to generate stimulus electrical pulses and deliver the stimulus electrical pulses to the areas of interest in the patient's brain through electrode(s) 370. Stimulator 360 may include, for example, a waveform generator, such as a digital-to-analog converter or a frequency synthesizer, for generating the desired electrical pulses to regulate the patient's brain activities.

In one embodiment, movable radiopaque symbols may be used as the reconfigurable radiological indictors. The one or more reconfigurable radiological indictors may be shifted or rotated in or out of the path of the X-ray or the field of view of the fluoroscope. For example, a radiopaque symbol (e.g., a small disk, a character, or a particular shape made of metal or some other material that can block X-rays) may be moved by an indicator controller, such as a small magnetic, electrostatic, or electromechanical actuator, controlled by the controller of the DBS system based on the electrical signals collected by the electrode. For example, in some embodiments, the actuator may be configured to electrically apply a magnetic field in the controller of the DBS system, which may rotate the radiological indictors to align with the orientation of the magnetic field. In some embodiments, a micro-motor, such as a microelectromechanical system (MEMS) motor, may be used to shift or rotate the radiological indictors.

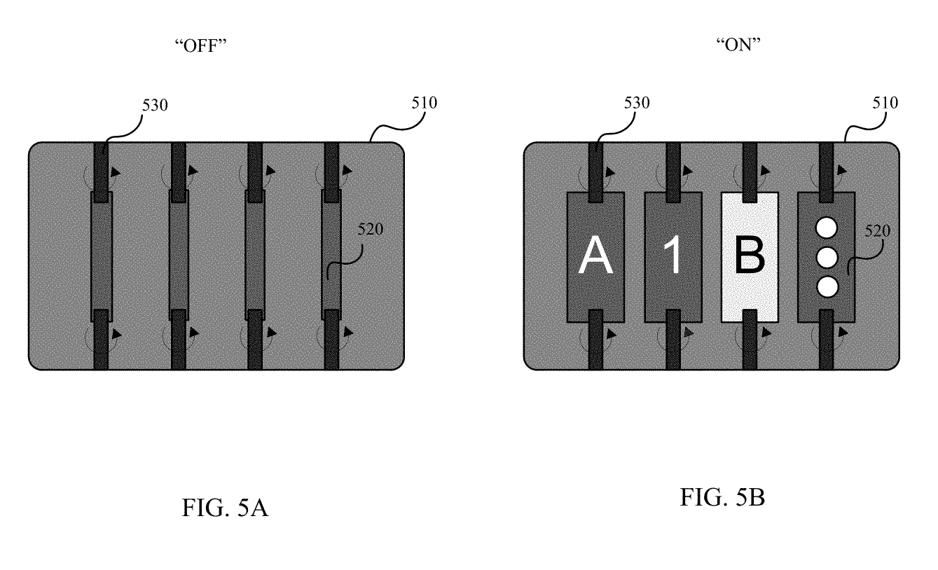

FIG. 5A illustrates example radiological indicators set in “OFF” states, according to certain aspects of the present disclosure. FIG. 5B illustrates example radiological indicators set in “ON” states, according to certain aspects of the present disclosure. As shown in FIGS. 5A and 5B, a controller of the DBS system, such as controller 250 or 300, may include an enclosure 510, which may be vacuumed or may be filled with a fluid, such as air or a liquid. One or more radiological indicators 520 (e.g., radiopaque indicators) may be disposed in enclosure 510. Each radiological indicator 520 may show a letter, number, or shape when being set in the “ON” state and being viewed using an X-ray imaging system. Each radiological indicator 520 may be coupled to a pivot 530 and may turn with pivot 530, where pivot 530 and/or radiological indicator 520 may be rotated by a small actuator, such as a magnetic, electrostatic, or electromechanical actuator. Alternatively, each radiological indicator 520 may be loosely coupled to a shaft and may turn around the shaft by a small actuator, such as a magnetic, electrostatic, or electromechanical actuator.

For example, in some embodiments, before a predetermined condition is met (e.g., when electrical signals associated with tremors are not detected, which may indicate that the tip of the electrode has not reached the area of interest or is moved away from the area of interest), radiological indicator 520 corresponding to the electrode may be set to an “OFF” state as shown in FIG. 5A. In the “OFF” state, the indicator controller may move radiological indicator 520 away from the path or rotate the radiological indicator 520 such that the larger surface area of radiological indicator 520 is substantially parallel to the propagation direction of the X-ray so that it does not block the path from the X-ray source to the X-ray sensor, and radiological indicator 520 may be seen as a dark area in the X-ray image, and no letter, number, or shape may be seen in the X-ray image. In some cases, the edges of radiological indicator 520 may still be seen, but they may generally appear as thin lines and may not show the letter, number, or shape associated with radiological indicator 520. Radiological indicator(s) 520 may include radiopaque materials, such as gold, aluminum, stainless steel, titanium, or some radiopaque polymer materials.

When a predetermined condition is met (e.g., when electrical signals associated with tremors are detected, which may indicate that the tip of the electrode is located at the area of interest), radiological indicator 520 corresponding to the electrode may be set to an “ON” state as shown in FIG. 5B. In the “ON” state, the indicator controller may move or rotate radiological indicator 520 corresponding to the electrode to block the X-ray path from the X-ray source to the X-ray sensor. For example, radiological indicator 520 may be rotated such that the larger surface area of radiological indicator 520 is substantially perpendicular to the propagation direction of the X-ray, and radiological indicator 520 at least partially blocks X-rays from the X-ray source to the X-ray sensor. Thus, the image of radiological indicator 520 may become “white” in the corresponding area on the X-ray image. As such, a symbol associated with radiological indicator 520, such as a letter, number, or shape, may become visible on the screen of the fluoroscope.

In this way, the status of the DBS system or the location of the electrode indicated by radiological indicator 520 may be read by an X-ray imaging system. It is noted that although the radiological indicators in the above-described embodiments may be set to the “ON” state when the predetermined condition is met and may be set to the “OFF” state when the predetermined condition is not met, in some embodiments, the radiological indicators may be set to the “OFF” state when the predetermined condition is met, and may be set to the “ON” state when the predetermined condition is not met.

FIG. 5B also illustrates example radiopaque symbols of radiological indicators. For example, some radiological indicators may include a letter or numerical number that is formed of a radiopaque material, and thus the letter may become “white” in the X-ray image when the radiological indicator is set to the “ON” state. Some radiological indicators may include a body made of a radiopaque material with a cutout (or radiolucent) region in the shape of a letter or numerical number and the body of the radiological indicators other than the radiolucent region may become “white” in the X-ray images when the radiological indicator is set to the “ON” state. In some cases, a radiological indicator may include any arbitrary radiopaque shape or radiolucent shape, such as dots, bars, curves, etc.

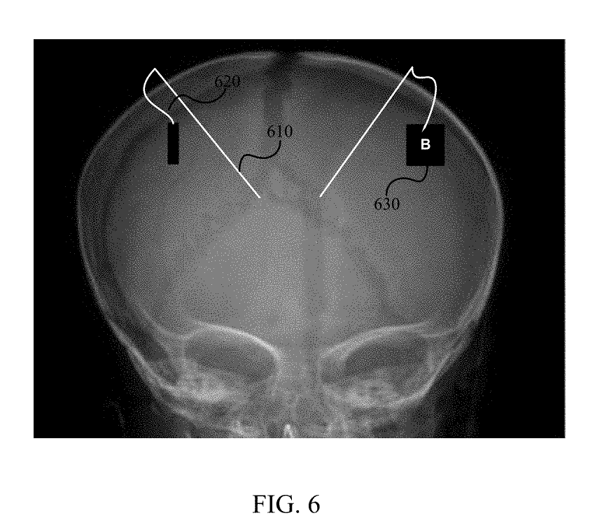

FIG. 6 is an illustrative X-ray image of a patient's head with an implanted DBS implant system, according to certain aspects of the present disclosure. The DBS implant system may include implantations on both the left and right sides of the brain. The example X-ray image shows electrodes 610, extension wires 620, and radiological indicators 630 of the DBS implant system on both sides of the brain. The radiological indicator on the right side of the brain (left side in the x-ray image) is in the “OFF” state, which may indicate, for example, electrode 610 on the right side of the brain may not be properly located at an area of interest or some other components in the right half of the DBS implant system may not function properly. FIG. 6 shows that the radiological indicator on the left side of the brain is in the “ON” state, which may indicate, for example, electrode 610 on the left side of the brain is located at an area of interest and other components in the left half of the DBS implant system function properly.

In some embodiments, the radiological indicator may be moved, for example, horizontally and/or vertically. In some embodiments, the radiological indicator may be rotated. In some embodiments, a visible indicator may indicate that the electrode is in a proper location. In some embodiments, a visible indicator may indicate that the electrode is not in a proper location. In some embodiments, one or more radiological indicators may be used to indicate the status of one electrode, where each radiological indicator may indicate one condition of the electrode or the radiological indicators may be used jointly to indicate a condition of the electrode, such as using a binary code. In some embodiments, the radiological indicator may be reconfigured to multiple states, rather than just the “ON” and “OFF” states. For example, the radiological indicator may be set to different states when being rotated by different angles or directions. In some embodiments, two or more radiological indicators may be set to jointly represent different states.

In some embodiments, an indicator including a material that can change its response to X-ray when a voltage is applied to it may be used as the radiological indicator. For example, the material may be transparent to X-rays when no voltage is applied to it, but may absorb or scatter X-rays when a voltage is applied to it. When the electrical signals collected by the electrode indicate that a predetermined condition is met, the indicator controller may apply a voltage to the radiological indicator, which may absorb or scatter X-rays, and thus become radiopaque to provide the indication in the X-ray image. In some embodiments, the material may be radiopaque when no voltage is applied to it, but may become radiolucent when a voltage is applied to it.

In this way, during the implantation procedure, the controller of the DBS system may receive electrical signals collected by the electrode and set the radiological indicator(s) accordingly in real-time. The radiological indicator(s) may be seen on the fluoroscope. When the indicator(s) indicate that a characteristic pattern associated with a disease is detected and thus the electrode is in the area of interest, the electrode may be fixed in place and the cranium may be closed. At any time instant, if the electrode is moved such that the characteristic pattern is no longer detected, immediate feedback may be provided to the surgeon through the indicator(s), and a corrective action may be taken to reposition the electrode before the surgery is complete, which may result in a higher success rate for DBS implantation. The radiological indicators included in the controller of the DBS system may also be used during the lifetime of the DBS system to indicate whether the electrode remains in the area of interest or whether other components of the DBS system (e.g., any circuit in the controller as shown in FIG. 3, the extension wire, or any connection point) operate properly, when viewed using an X-ray imaging system.

### III. Ultrasonic Indicators

In some embodiments, an implant device may be implanted in another portion of a body, such as the abdomen, where an ultrasonic imager may be used to monitor the portion of the body during the implantation procedure. The ultrasonic imager may include an ultrasonic emitter or a (phased) transmitter array for transmitting ultrasonic pulses to the body, and an ultrasonic detector or a (phased) detector array for detecting the ultrasonic pulses reflected or otherwise returned by different parts of the body. As in the embodiments described above, the implant device may include a controller, an electrode, and an insulated extension wire connecting the controller and the electrode. The controller may include or be connected to an indicator that can be configured to be visible to the ultrasonic imager.

Various types of indicator may be used to provide information regarding the condition or status of the electrode or the implant device during or after the implantation procedure, when viewed using the ultrasonic imager. For example, in some embodiments, the indicator may be similar to the radiological indicator described above, but may be made of materials opaque to ultrasonic signals. The indicator may be shifted or rotated to be moved into or out of the path of the ultrasonic signals from the ultrasonic imager by various actuators, such as actuators similar to the actuators described above.

During ultrasound imaging, an object being scanned may return an echo signal that varies with the surface compliance of the scanned object. A hard surface may return a strong echo signal, while a soft surface may return a weak echo signal. In some embodiments, a solenoid controlled by the controller may be placed under a surface of a controller or other parts of the implant device to be used as an indicator. Depending on the electrical signals collected by the electrode implanted in the body, the controller may, for example, control the solenoid such that the solenoid may extend to touch the surface of the implant device or the controller and firmly support a septum of a flexible material (e.g., rubber). A strong echo signal may thus be received by the ultrasonic imager. When the electrical signals detected by the electrode indicate a different condition of the electrode, the controller may control the solenoid such that the solenoid may retract and may not touch the surface of the implant device or the controller, thus allowing the septum to resume its normal flexibility, and an attenuated echo signal may then be received by the ultrasonic imager.

In some embodiments, an ultrasonic transponder may be used as the indicator. The ultrasonic transponder may receive an ultrasonic pulse from an external ultrasonic emitter using an ultrasonic sensor and transmit a different ultrasonic pulse using an ultrasonic emitter. When, for example, a characteristic electrical pattern associated with the disease to be treated is received by a controller from an electrode implanted in the body, the controller may cause the emitter of the transponder to generate a strong ultrasonic pulse. The echo from the surface of the implant device or the controller may thus be amplified by the strong ultrasonic pulse to provide a stronger return signal. The transponder may be turned off when, for example, the characteristic electrical pattern is not detected, such that a normal return signal may be generated, for example, by the surface of the implant device or controller. The difference between the two return signals may indicate a change of condition (e.g., location or connection) of the electrode. In various embodiments, the sensor and the emitter of the transponder may be implemented using a same physical device or different physical devices. The transponder may be connected to or integrated into the controller of the implant device.

In some embodiments, the ultrasonic transponder may also act as an indicator by delaying the returned signals to convey more complex information. The sensor of the ultrasonic transponder may receive ultrasonic pulses from the emitter of the ultrasonic imager. When, for example, a characteristic electrical pattern associated with the disease to be treated is received by the controller of the implant device from an electrode implanted in the patient's body, the controller may cause the emitter of the transponder to wait a specified amount of time (such as several microseconds) and then generate one or more ultrasonic pulses. The normal echo from the surface of the implant device (or the controller) may thus be augmented by the additional pulses from the transponder to provide a stronger return pulse or multiple return pulses. Since an ultrasound imager can use delays of the return signals to determine the distances of the locations where the return signals are generated, the delayed ultrasonic pulses from the transponder may be viewed by the ultrasonic imager as being reflected from locations further away from the ultrasonic imager. If more than one delayed ultrasonic pulse is transmitted by the transponder, a series of lines distal to the ultrasonic imager may be seen in the ultrasonic image. The series of lines may be used to encode more complex information than an on/off signal could.

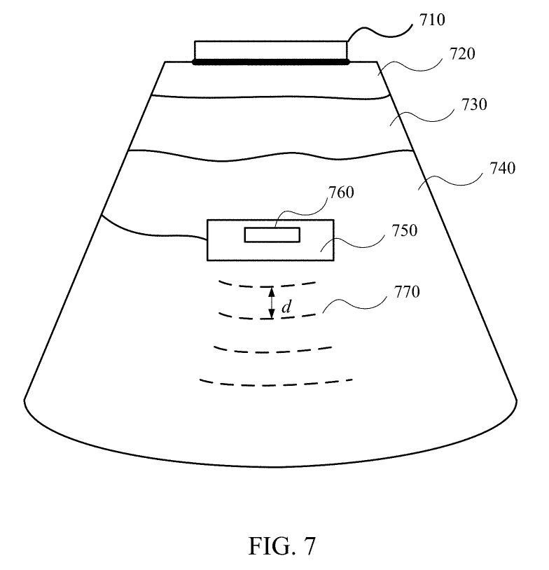

FIG. 7 illustrates an example implant device in the abdomen of a patient, according to certain aspects of the present disclosure. The implant device may include a controller 750 that may be implanted in any portion of the abdomen area, such as dermis 720, subcutaneous layer 730, or area 740 under subcutaneous layer 730. During or after the implantation of the implant device, an ultrasonic imaging system including a transceiver 710 may be used to monitoring the physical location and/or status of the implant device, such as the location of an electrode of the implant device. As described above, transceiver 710 may include an ultrasonic emitter or a (phased) transmitter array for transmitting ultrasonic pulses to the body, and an ultrasonic detector or a (phased) ultrasonic detector array for detecting the ultrasonic pulses returned by different parts of the body.

In some embodiments, controller 750 may include or be coupled to a reconfigurable ultrasonic indicator 760 for providing information regarding the status of the implant device, such as whether the electrode of the implant device is at a desired location. As described above, in some embodiments, ultrasonic indicator 760 may include a solenoid controlled by controller 750. Based on the electrical signals collected by the electrode implanted in the body, controller 750 may control the solenoid to indicate different status of the implant device. For example, when the electrical signals detected by the electrode indicate that the electrode is not in the desired location, controller 750 may activate the solenoid such that the solenoid may extend to touch the surface of controller 750 and firmly supports a flexible material (e.g., rubber) at a surface of controller 750. A strong echo signal may thus be received by transceiver 710 of the ultrasonic imaging system. When the electrical signals detected by the electrode indicate a different condition of the electrode, the controller may deactivate the solenoid such that the solenoid may retract and may not touch the flexible surface of controller 750, thus allowing the flexible surface of controller 750 to resume its normal flexibility. An attenuated echo signal may then be generated at the flexible surface and be received by transceiver 710 of the ultrasonic imaging system.

In some implementations, ultrasonic indicator 760 may include an ultrasonic transponder. An ultrasonic sensor of the transponder may receive an ultrasonic pulse from the ultrasonic emitter of transceiver 710, and an emitter of the transponder may transmit a return ultrasonic pulse in response to receiving the ultrasonic pulses from transceiver 710. The return ultrasonic pulse may have, for example, a different amplitude or delay to indicate a different status of the implant device. In some implementations, controller 750 may cause the emitter of the transponder to generate a strong (high amplitude) ultrasonic pulse when, for example, the implant device does not function properly. The echo from the surface of controller 750 may thus be amplified by the strong ultrasonic pulse to provide a stronger return signal. The transponder may be turned off when, for example, the implant device functions properly, such that a normal return signal from the surface of controller 750 may be generated. The difference between the two return signals may indicate a change of status of the implant device, such as the change of position of the electrode of the implant device during or after implantation. Thus, the amplitude of the return signal may indicate the status of the implant device.

In some implementations, controller 750 may cause the emitter of the ultrasonic transponder to generate delayed return signals to convey more complex information. For example, controller 750 may cause the emitter of the ultrasonic transponder to generate a series of ultrasonic return pulses, where the delay between two consecutive return pulses may be a specified value (e.g., several microseconds). Since an ultrasound imaging system can use time delays to determine distances, the delayed return signals may be viewed by the ultrasound imaging system as being reflected at locations (e.g., lines 770) further from transceiver 710 compared with controller 750. If more than one delayed return pulse is generated by the transponder, a series of lines 770 distal to transceiver 710 may be seen in the ultrasonic image. The series of lines may be used to encode more complex information than an on/off signal could. In some implementations, a different distance d between two or more lines 770 (caused by a different delay between two return pulses) may be used to indicate a different status of the implant device.

### IV. Example Methods

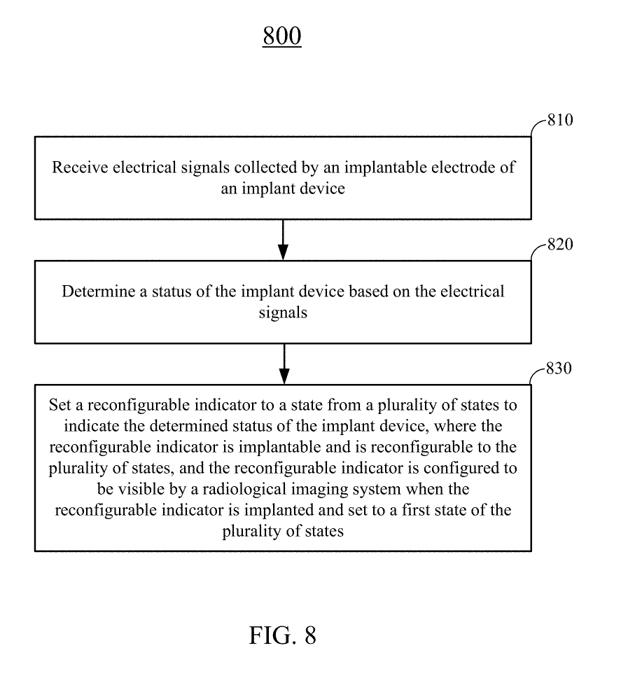

FIG. 8 is a flow chart 800 illustrating an example method for indicating a status of an implant device, such as a DBS system, according to some aspects of the present disclosure. At block 810, a controller of the implant device, such as controller 250 of FIG. 2A, or controller 300 of FIG. 3, may receive electrical signals collected by an electrode implanted in a patient's body. For example, the electrode may be implanted in the brain of the patient, where the tip of the electrode may be intended to locate in an area of interest inside the brain, such as the thalamic area, the subthalamic nucleus (STN), the external globus pallidus (GPe), or the medial globus pallidus internus (GPI) of a brain, for sensing electrical activities associated with, for example, Parkinson's disease, and applying electrical pulses to regulate the electrical activities of brain neurons.

At block 820, the controller may determine a status of the implant device, such as a location of the electrode in the body, based on the collected electrical signals. For example, the controller may compare the electrical signals collected by the electrode with typical electrical signals measured from a patient suffering from Parkinson's disease or search for a characteristic electrical pattern associated with Parkinson's disease in the collected electrical signals. If the tip of the electrode is located in the area of interest, the collected electrical signals may be similar to certain electrical signals measured from a patient suffering from Parkinson's disease or may include a characteristic electrical pattern associated with Parkinson's disease. The controller may thus determine whether the tip of the electrode is located in the area of interest based on the collected electrical signals. In some embodiments, even if the tip of the electrode is located in an area of interest, the electrical signals collected by the tip of the electrode may not be delivered to the controller (e.g., due to faulty electrical connection(s) between two or more components of the implant device), or may be misprocessed by a malfunctioning controller that may determine that the tip of the electrode is not located in the area of interest.

At block 830, the controller may set a reconfigurable indicator to a state from a plurality of states to indicate the determined status of the implant device, such as the location of the electrode. The reconfigurable indicator is implantable and may be dynamically reconfigurable to the plurality of states, where the reconfigurable indicator is configured to be visible by an imaging system when the reconfigurable indicator is implanted and set to at least a first state of the plurality of states. Each state of the plurality of states of the reconfigurable indicator may correspond to a different status of the implant device when the implant device is viewed using the imaging system, such as an X-ray imaging system. An image of the reconfigurable indicator formed by the imaging system may be different in each state of the plurality of states. For example, if the controller determines that the electrode is properly located in the area of interest, the controller may set the reconfigurable indicator to be visible by the X-ray imaging system as described above in the present disclosure. As described above, the reconfigurable indicator may not be correctly set to indicate that the tip of the electrode is located in the area of interest if some other components of the implant device are at fault. As such, the reconfigurable indicator may also indicate the operational status of the overall implant device.

It is noted that even though FIG. 8 describes the operations as a sequential process, some of the operations may be performed in parallel or concurrently. In addition, the order of the operations may be rearranged. An operation may have additional steps not included in the figure. Some operations may be optional, and thus may be omitted in various embodiments. Some operations described in one block may be performed together with operations described at another block. Furthermore, embodiments of the methods may be implemented in hardware, software, firmware, middleware, microcode, hardware description languages, or any combination thereof.

### V. Example Computing Systems

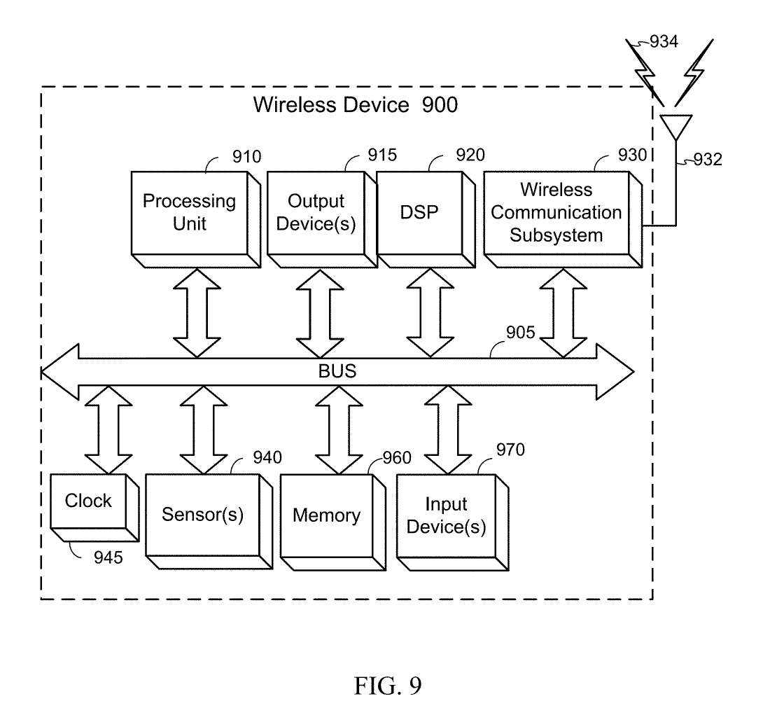

FIG. 9 illustrates an embodiment of a wireless device 900, which can be utilized as described herein above. For example, wireless device 900 can be used in controller 130 of FIG. 1, controller 250 of FIG. 2A, or controller 300 of FIG. 3. It should be noted that FIG. 9 is meant only to provide a generalized illustration of various components, any or all of which may be utilized as appropriate. It can be noted that, in some instances, components illustrated by FIG. 9 can be localized to a single physical device and/or distributed among various devices, which may be disposed at different physical locations. As such, components may vary from embodiment to embodiment.

Wireless device 900 is shown comprising hardware elements that can be electrically coupled via a bus 905 (or may otherwise be in communication, as appropriate). The hardware elements may include a processing unit(s) 910 which can include without limitation one or more general-purpose processors, one or more special-purpose processors (such as digital signal processing (DSP) chips, graphics acceleration processors, application specific integrated circuits (ASICs), and/or the like), and/or other processing structure or means, which can be configured to perform one or more of the methods described herein, such as determining whether an electrode is at an area of interest. As shown in FIG. 9, some embodiments may have a separate DSP 920, depending on desired functionality. Wireless device 900 also can include one or more input devices 970, which can include without limitation a touch pad, button(s), dial(s), switch(es), and/or the like; and one or more output devices 915, which can include without limitation light emitting diodes (LEDs), speakers, and/or the like.

Wireless device 900 might also include a wireless communication subsystem 930, which can include without limitation a wireless communication device, and/or a chipset (such as a Bluetooth device, an International Electrical and Electronics Engineers (IEEE) 802.11 device (e.g., a device utilizing one or more of the 902.11 standards described herein), an IEEE 802.15.4 device, a WiFi device, a WiMax device, cellular communication facilities, etc.), and/or the like. Wireless communication subsystem 930 may permit data to be exchanged with a network, wireless access points, other computer systems, and/or any other electronic devices described herein, such as a mobile device or a remote controller. The communication can be carried out via one or more wireless communication antenna(s) 932 that send and/or receive wireless signals 934. In various embodiments, wireless communication subsystem 930 may be used to receive commands from a patient to turn on or off the implant device, or to receive commands from medical personnel to set parameters for the implant device, such as parameters for the stimulator.

Depending on desired functionality, wireless communication subsystem 930 can include separate transceivers to communicate with antennas of base transceiver stations and other wireless devices and access points as described above, which may include communicating with different data networks and/or network types, such as wireless wide-area networks (WWANs), wireless local area networks (WLANs), or wireless personal area networks (WPANs). A WWAN may be a network using any air interface technology, for example, a code division multiple access (CDMA) network, a Time Division Multiple Access (TDMA) network, a Frequency Division Multiple Access (FDMA) network, an Orthogonal Frequency Division Multiple Access (OFDMA) network, a Single-Carrier Frequency Division Multiple Access (SC-FDMA) network, a WiMax (IEEE 802.16), and so on. A CDMA network may implement one or more radio access technologies (RATs) such as cdma2000, W-CDMA, and so on. Cdma2000 includes IS-95, IS-2000, and/or IS-856 standards. A TDMA network may implement GSM, Digital Advanced Mobile Phone System (D-AMPS), or some other RATs. An OFDMA network may employ long-term evolution (LTE), LTE Advanced, and so on. LTE, LTE Advanced, GSM, and W-CDMA are described in documents from 3GPP. Cdma2000 is described in documents from a consortium named “3rd Generation Partnership Project 2” (3GPP2). 3GPP and 3GPP2 documents are publicly available. A WLAN may be an IEEE 802.11x network. A WPAN may be a Bluetooth network, an IEEE 802.15x, or some other type of network.

Wireless device 900 may include a clock 945 on bus 905, which can generate a signal to synchronize various components on bus 905. Clock 945 may include an inductor-capacitor (LC) oscillator, a crystal oscillator, a ring oscillator, a digital clock generator such as a clock divider or clock multiplexer, a phase locked loop, or other clock generator. Clock 945 may be synchronized (or substantially synchronized) with corresponding clocks on other wireless devices for data communication. Clock 945 may be driven by wireless communication subsystem 930, which may be used to synchronize clock 945 of wireless device 900 to one or more other devices. Clock 945 may be used as the time base or reference for generating periodic stimulus pulses.

Wireless device 900 can further include sensor(s) 940. Such sensors can include, without limitation, one or more accelerometer(s), gyroscope(s), pressure sensor(s), proximity sensor(s), light sensor(s), and the like. Some or all of sensor(s) 940 can be utilized, among other things, for positioning or motion detection.

Wireless device 900 may further include and/or be in communication with a memory 960. Memory 960 may include any non-transitory storage device, and may include, without limitation, local and/or network accessible storage, a disk drive, a drive array, an optical storage device, a solid-state storage device, such as a random access memory (RAM), and/or a read-only memory (ROM), which can be programmable, flash-updateable, and/or the like. Such storage devices may be configured to implement any appropriate data stores, including without limitation, various file systems, database structures, and/or the like. For instance, memory 960 may include a database (or other data structure) configured to store detected electrical signals, example electrical signals measured from patients suffering from, for example, Parkinson's disease, electrical pulses to be applied through the electrode, and various parameters of the controller, as described in embodiments herein.

Memory 960 of wireless device 900 also can comprise software elements (not shown), including an operating system, device drivers, executable libraries, and/or other code, such as one or more application programs, which may comprise computer programs provided by various embodiments, and/or may be designed to implement methods, and/or configure systems, provided by other embodiments, as described herein. Merely by way of example, one or more procedures described with respect to the functionality discussed above, such as the method shown in FIG. 8 might be implemented as code and/or instructions that can be stored or loaded in memory 960 and be executed by wireless device 900, a processing unit within wireless device 900, and/or another device of a wireless system. In an aspect, such code and/or instructions can be used to configure and/or adapt a general purpose computer (or other device) to perform one or more operations in accordance with the described methods.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

With reference to the appended figures, components that can include memory can include non-transitory machine-readable media. The terms “machine-readable medium” and “computer-readable medium” as used herein refer to any storage medium that participates in providing data that causes a machine to operate in a specific fashion. In embodiments provided hereinabove, various machine-readable media might be involved in providing instructions/code to processors and/or other device(s) for execution. Additionally or alternatively, the machine-readable media might be used to store and/or carry such instructions/code. In many implementations, a computer-readable medium is a physical and/or tangible storage medium. Such a medium may take many forms, including, but not limited to, non-volatile media, volatile media, and transmission media. Common forms of computer-readable media include, for example, magnetic and/or optical media, punch cards, paper tape, any other physical medium with patterns of holes, a RAM, a programmable read-only memory (PROM), an erasable programmable read-only memory (EPROM), a FLASH-EPROM, any other memory chip or cartridge, a carrier wave as described hereinafter, or any other medium from which a computer can read instructions and/or code.

The methods, systems, and devices discussed herein are examples. Various embodiments may omit, substitute, or add various procedures or components as appropriate. For instance, features described with respect to certain embodiments may be combined in various other embodiments. Different aspects and elements of the embodiments may be combined in a similar manner. The various components of the figures provided herein can be embodied in hardware and/or software. Also, technology evolves and, thus, many of the elements are examples that do not limit the scope of the disclosure to those specific examples.

It has proven convenient at times, principally for reasons of common usage, to refer to such signals as bits, information, values, elements, symbols, characters, variables, terms, numbers, numerals, or the like. It should be understood, however, that all of these or similar terms are to be associated with appropriate physical quantities and are merely convenient labels. Unless specifically stated otherwise, as is apparent from the discussion above, it is appreciated that throughout this specification discussions utilizing terms such as “processing,” “computing,” “calculating,” “determining,” “ascertaining,” “identifying,” “associating,” “measuring,” “performing,” or the like refer to actions or processes of a specific apparatus, such as a special purpose computer or a similar special purpose electronic computing device. In the context of this specification, therefore, a special purpose computer or a similar special purpose electronic computing device is capable of manipulating or transforming signals, typically represented as physical electronic, electrical, or magnetic quantities within memories, registers, or other information storage devices, transmission devices, or display devices of the special purpose computer or similar special purpose electronic computing device.

Those of skill in the art will appreciate that information and signals used to communicate the messages described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Terms “and,” “or,” and “an/or,” as used herein, may include a variety of meanings that also is expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, B, C, AB, AC, BC, AA, AAB, AABBCCC, etc.

Reference throughout this specification to “one example,” “an example,” “certain examples,” or “exemplary implementation” means that a particular feature, structure, or characteristic described in connection with the feature and/or example may be included in at least one feature and/or example of claimed subject matter. Thus, the appearances of the phrase “in one example,” “an example,” “in certain examples,” “in certain implementations,” or other like phrases in various places throughout this specification are not necessarily all referring to the same feature, example, and/or limitation. Furthermore, the particular features, structures, or characteristics may be combined in one or more examples and/or features.

Some portions of the detailed description included herein may be presented in terms of algorithms or symbolic representations of operations on binary digital signals stored within a memory of a specific apparatus or special purpose computing device or platform. In the context of this particular specification, the term specific apparatus or the like includes a general-purpose computer once it is programmed to perform particular operations pursuant to instructions from program software. Algorithmic descriptions or symbolic representations are examples of techniques used by those of ordinary skill in the signal processing or related arts to convey the substance of their work to others skilled in the art. An algorithm is here, and generally, considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared, or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining,” or the like refer to actions or processes of a specific apparatus, such as a special purpose computer, special purpose computing apparatus or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

In the preceding detailed description, numerous specific details have been set forth to provide a thorough understanding of claimed subject matter. However, it will be understood by those skilled in the art that claimed subject matter may be practiced without these specific details. In other instances, methods and apparatuses that would be known by one of ordinary skill have not been described in detail so as not to obscure claimed subject matter. Therefore, it is intended that claimed subject matter not be limited to the particular examples disclosed, but that such claimed subject matter may also include all aspects falling within the scope of appended claims, and equivalents thereof.

For an implementation involving firmware and/or software, the methodologies may be implemented with modules (e.g., procedures, functions, and so on) that perform the functions described herein. Any machine-readable medium tangibly embodying instructions may be used in implementing the methodologies described herein. For example, software codes may be stored in a memory and executed by a processor unit. Memory may be implemented within the processor unit or external to the processor unit. As used herein the term “memory” refers to any type of long term, short term, volatile, nonvolatile, or other memory and is not to be limited to any particular type of memory or number of memories, or type of media upon which memory is stored.

If implemented in firmware and/or software, the functions may be stored as one or more instructions or code on a computer-readable storage medium. Examples include computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise RAM, ROM, EEPROM, compact disc read-only memory (CD-ROM) or other optical disk storage, magnetic disk storage, semiconductor storage, or other storage devices, or any other medium that can be used to store desired program code in the form of instructions or data structures and that can be accessed by a computer; disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

In addition to storage on computer-readable storage medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims. That is, the communication apparatus includes transmission media with signals indicative of information to perform disclosed functions. At a first time, the transmission media included in the communication apparatus may include a first portion of the information to perform the disclosed functions, while at a second time the transmission media included in the communication apparatus may include a second portion of the information to perform the disclosed functions.

What is claimed is:

1. An implant device comprising:
a reconfigurable indicator; and
a controller comprising or coupled to the reconfigurable indicator,
wherein the reconfigurable indicator is implantable, and is reconfigurable to a plurality of states by the controller; and
wherein the reconfigurable indicator is configured to be visible by an imaging system when the reconfigurable indicator is implanted and set to a first state of the plurality of states.

2. The implant device of claim 1, wherein the reconfigurable indicator, when set to the first state, is visible by a radiological imaging system or an ultrasonic imaging system.

3. The implant device of claim 1, wherein:
the imaging system comprises an X-ray imaging system; and
the reconfigurable indicator comprises a symbol comprising a radiopaque material, wherein, in the first state of the plurality of states, the symbol is configured to at least partially block X-rays from the X-ray imaging system.

4. The implant device of claim 3, wherein the symbol on the reconfigurable indicator comprises at least one of a letter, a number, or a figure.

5. The implant device of claim 3, wherein the controller comprises two or more reconfigurable indicators, the two or more reconfigurable indicators configurable to, in combination, indicate a status of the implant device.

6. The implant device of claim 3, wherein, in a second state of the plurality of states, the symbol on the reconfigurable indicator is configured to be invisible by the X-ray imaging system.

7. The implant device of claim 1, wherein:
each state of the plurality of states of the reconfigurable indicator corresponds to a different status of the implant device; and
the reconfigurable indicator, when set to a different state of the plurality of states, is configured to cause a different image of the reconfigurable indicator to be formed by the imaging system.

8. The implant device of claim 7, wherein the controller comprises instructions stored thereon, the instructions, when executed by the controller, causing the controller to:
receive electrical signals from an implantable electrode of the implant device;
determine a location of the implantable electrode based on the electrical signals; and
set the reconfigurable indicator to a state of the plurality of states based on the determined location of the implantable electrode.

9. The implant device of claim 8, wherein the instructions, when executed by the controller, further cause the controller to:
determine a status of the implant device based on the electrical signals; and
set the reconfigurable indicator to indicate the determined status of the implant device.

10. The implant device of claim 8, wherein:
the implant device comprises a deep brain stimulation (DBS) device and an implantable electrode;
the implantable electrode is configured to collect electrical signals in a brain of a person; and
the instructions, when executed by the controller, further cause the controller to:
detect a characteristic pattern of electrical pulses associated with a disease from the electrical signals collected by the implantable electrode; and
set the reconfigurable indicator to a state of the plurality of states to indicate that the implantable electrode is in an area of interest in the brain of the person.

11. The implant device of claim 10, wherein the disease comprises Parkinson's disease.

12. The implant device of claim 1, wherein the controller further comprises an actuator, the actuator configured to shift or rotate the reconfigurable indicator to set the reconfigurable indicator to the plurality of states.

13. The implant device of claim 12, wherein the actuator comprises at least one of a magnetic, electrostatic, or electromechanical actuator.

14. The implant device of claim 1, wherein the controller is configured to be implantable under a scalp of a person.

15. The implant device of claim 1, wherein the controller comprises an enclosure and the reconfigurable indicator is positioned within the enclosure.

16. The implant device of claim 15, wherein the enclosure is vacuumed or is filled with a fluid.

17. The implant device of claim 1, wherein the imaging system comprises a fluoroscope or an ultrasonic imager.

18. The implant device of claim 1, wherein:
the imaging system comprises an ultrasonic imaging system; and
the reconfigurable indicator comprises a solenoid, wherein, in the first state of the plurality of states, the solenoid is configured to extend and support a surface of the controller.

19. The implant device of claim 1, wherein:
the imaging system comprises an ultrasonic imaging system; and
the reconfigurable indicator comprises an ultrasonic transponder, wherein, in the first state of the plurality of states, the ultrasonic transponder is configured to:
detect an incoming ultrasonic signal from the ultrasonic imaging system; and
in response to detecting the incoming ultrasonic signal, transmit a return ultrasonic signal.

20. The implant device of claim 19, wherein the return ultrasonic signal has a predetermined amplitude.

21. The implant device of claim 19, wherein the return ultrasonic signal is transmitted at a predetermined time after the incoming ultrasonic signal is received by the ultrasonic transponder.

22. The implant device of claim 19, wherein:
the return ultrasonic signal comprises two or more ultrasonic pulses; and
a delay between two of the two or more ultrasonic pulses is a predetermined value.

23. A method for indicating a status of an implant device, the method comprising:
receiving electrical signals collected by an implantable electrode;
determining the status of the implant device based on the electrical signals; and
setting a reconfigurable indicator to a state from a plurality of states to indicate the determined status of the implant device, wherein the reconfigurable indicator is implantable and is reconfigurable to the plurality of states, and wherein the reconfigurable indicator is configured to be visible by an imaging system when the reconfigurable indicator is implanted and set to a first state of the plurality of states.

24. The method of claim 23, wherein:
each state of the plurality of states of the reconfigurable indicator corresponds to a different status of the implant device; and
the reconfigurable indicator, when set to a different state of the plurality of states, is configured to cause a different image of the reconfigurable indicator to be formed by the imaging system.

25. The method of claim 23, wherein determining the status of the implant device comprises determining a location of the implantable electrode.

26. The method of claim 23, wherein setting the reconfigurable indicator to the state comprises shifting or rotating the reconfigurable indicator using an actuator.

27. The method of claim 26, wherein the actuator comprises a magnetic, electrostatic, or electromechanical actuator.

28. The method of claim 23, further comprising:
reading, using the imaging system, the determined status of the implant device indicated by the reconfigurable indicator,
wherein the imaging system comprises an X-ray imaging system; and
wherein the reconfigurable indicator comprises a symbol comprising a radiopaque material, wherein, in the first state of the plurality of states, the symbol is configured to at least partially block X-rays from the X-ray imaging system.

29. The method of claim 23, wherein determining the status of the implant device based on the electrical signals comprises detecting a characteristic pattern of electrical pulses associated with a disease from the electrical signals.

30. The method of claim 29, wherein the disease comprises Parkinson's disease.

31. The method of claim 23, wherein:
the imaging system comprises an ultrasonic imaging system;
the reconfigurable indicator comprises a solenoid; and
setting the reconfigurable indicator to the first state comprises causing the solenoid to extend.

32. The method of claim 23, wherein:
the imaging system comprises an ultrasonic imaging system;
the reconfigurable indicator comprises an ultrasonic transponder; and
setting the reconfigurable indicator to the first state comprises causing the ultrasonic transponder to:
detect an incoming ultrasonic signal from the ultrasonic imaging system; and
in response to detecting the incoming ultrasonic signal, transmit a return ultrasonic signal comprising one or more ultrasonic pulses.

33. An apparatus comprising:
means for receiving electrical signals collected by an implantable electrode;
means for determining a status of the apparatus based on the electrical signals; and
means for indicating the determined status of the apparatus,
wherein the means for indicating the determined status of the apparatus is implantable and is reconfigurable to a plurality of states, each state of the plurality of states corresponding to a different status of the apparatus; and
wherein the means for indicating the determined status of the apparatus is configured to be visible by an imaging system when the means for indicating the determined status of the apparatus is implanted and set to a first state of the plurality of states.

34. The apparatus of claim 33, wherein the means for indicating the determined status of the apparatus, when set to a different state of the plurality of states, is configured to cause a different image of the means for indicating the determined status of the apparatus to be formed by the imaging system.

35. The apparatus of claim 33, wherein the means for determining the status of the apparatus based on the electrical signals comprises means for determining a location of the implantable electrode based on the electrical signals.

36. The apparatus of claim 33, wherein:
the means for indicating the determined status of the apparatus is movable and comprises a symbol comprising a radiopaque material;
the imaging system comprises an X-ray imaging system; and
the symbol is configured to at least partially block X-rays from the X-ray imaging system in the first state of the plurality of states.

37. A non-transitory computer-readable storage medium comprising machine-readable instructions stored thereon, the instructions, when executed by one or more processors, causing the one or more processors to:
receive electrical signals collected by an implantable electrode of an implant device;
determine a status of the implant device based on the electrical signals; and
set a reconfigurable indicator to a state from a plurality of states to indicate the determined status of the implant device, wherein the reconfigurable indicator is implantable and is reconfigurable to the plurality of states, and wherein the reconfigurable indicator is configured to be visible by an imaging system when the reconfigurable indicator is implanted and is set to a first state of the plurality of states.

38. The non-transitory computer-readable storage medium of claim 37, wherein determining the status of the implant device based on the electrical signals comprises detecting a characteristic pattern of electrical pulses associated with a disease from the electrical signals collected by the implantable electrod
